(19) United States
(12) Patent Application Publication
(10) Pub. No.: US 2024/0342258 A1
(43) Pub. Date: Oct. 17, 2024

(54) USING C1 ESTERASE INHIBITOR TO TREAT VIRAL INFECTION-RELATED SYMPTOMS

(71) Applicant: Pharming Intellectual Property B.V., Leiden (NL)

(72) Inventors: Bruno Giannetti, Leiden (NL); Anurag Relan, Leiden (NL); Isaac Melamed, Leiden (NL)

(73) Assignee: Pharming Intellectual Property B.V., Leiden (NL)

(21) Appl. No.: 18/577,708

(22) PCT Filed: Jul. 7, 2022
(86) PCT No.: PCT/EP2022/068912

§ 371 Date: Jan. 9, 2024

Related U.S. Application Data
(60) Provisional application No. 63/220,208, filed on Jul. 9, 2021.

(30) Foreign Application Priority Data
Jul. 9, 2021 (EP) 21184864.3

(51) Int. Cl. A61K 38/57; A61P 25/28

(52) U.S. Cl. CPC A61K 38/57; A61P 25/28

(57) ABSTRACT

The claimed invention relates to treatment of virus-related neurological symptoms, particularly methods for treating such symptoms by administering a complement inhibitor. The types of virus-related neurological symptoms that can be treated according to the invention include extreme fatigue, sensory loss such as loss of taste, cognitive changes, seizures, tremor, and stroke, and can be linked to infection by a coronavirus such as SARS-CoV-2. The invention includes administering complement inhibitor, which can be recombinant or purified C1 inhibitor.

### TECHNICAL FIELD

The disclosure relates to treatment of post-viral infection neurological symptoms, particularly methods for treating such symptoms by administering C1 esterase inhibitor (C1INH).

### BACKGROUND

The complement system is an integral part of the innate immune system and consists of a number of distinct plasma proteins that act as a first line of defense inducing an inflammatory response after opsonization of pathogens and dying cells (Walport 2001, N.E.J.M. 344:1058-1066; Walport 2001, N.E.J.M. 344:1140-1144). The complement system and particularly the lectin pathway has been found to interact with and be involved in the clearance of a number of viruses (Thielens 2002, Immunobiology 205:563-574; Kase et al. 1999, Immunology 97:385-392; Bibert et al. 2019, PLoS Pathol. 15; Schiela et al., Front. Immunology 9:2177; Bermejo-Jambrina et al., Front. Immunology 9:590). However, unregulated complement activation may play a role in the pathogenesis of symptoms induced by viruses including influenza and Severe Acute Respiratory Syndrome (SARS) corona viruses (CoV). For example, one experimental model suggests that the complement system may be implicated in SARS-CoV-induced lung disease via regulation of the systemic proinflammatory response. Complement deficient mice infected with SARS-CoV were affected less severely and showed a reduced lung involvement and lower local and systemic cytokine levels compared to control mice (Gralinski et al. 2018, mBio 9(5)). In line with that finding, inhibition of complement C5a signaling alleviated lung damage in a MERS-CoV mouse model (Jiang et al. 2018, Emerg. Microbes Infect. 7:77) and an influenza H7N9 monkey model (Sun et al. 2015, Clin. Infect. Dis. 60:586-595). The same was also observed in a mouse model of severe avian influenza infection when inhibiting the complement cascade at an early step (C3a) (Sun et al., 2013, Am. J. Resp. Cell Mol. Bio. 49:221-230).

Recent findings suggest a relationship between a post-infectious response and neurologic changes in patients with SARS-CoV-2 post-viral syndrome. The neurological symptoms described include extreme fatigue, sensory loss and taste, cognitive changes, seizures, tremor, and in rare instances, stroke. These changes may be a form of post-infectious immune response resulting in various neurological symptoms. In some instances, patients who have recovered from SARS-CoV-2 are experiencing post-viral fatigue syndrome. There remains a need in the field for a method of treating this post-viral syndrome. The invention disclosed herein meets that need with methods comprising administering a therapeutically effective amount of C1 esterase inhibitor (C1INH) to patients suffering from SARS-CoV-2 post-viral syndrome.

C1INH, a member of the serpin superfamily of serine-protease inhibitors, is an acute-phase protein that has manifold targets and biological functions, such as inhibition of leucocytes and interactions with endothelial cells and microorganisms. Further, C1INH is the natural and strong inhibitor of the classical and lectin pathway of complement (earliest step of complement activation) and factor XII and plasma kallikrein of the contact system. C1INH is encoded by a single gene (SERPING1) on chromosome 11 that consists of 8 exons and 7 introns. The entire genomic sequence is known and codes for a protein of 500 amino acids, including a 22 amino acid signal sequence (Carter P. et al. 1988, Euro. J. Biochem. 173; 163). Plasma C1INH is a glycoprotein of approximately 105 kDa and is heavily glycosylated, with up to 50% of its molecular mass consisting of carbohydrate.

Purified and recombinant forms of C1INH have been approved for use as, and are currently used as, a therapeutic. Currently, four C1INH therapeutic preparations are available, three of them plasma-derived (Cinryze®, Berinert®, and Haegarda®) and one recombinant, i.e., recombinant human C1INH (Ruconest®, Pharming, Leiden, The Netherlands). Recombinant human C1INH shares an identical protein structure with plasma-derived C1INH but has a different glycosylation pattern (containing abundant oligomannose residues), which is responsible for a shorter half-life than plasma-derived C1INH (3 h vs. 30 h) (Davis and Bernstein 2011, Clinical Risk Management 7:265-273; van Veen et al. 2012, J. Biotechnology 162:319-326). Despite the rather broad interference with several cascades and targets, major adverse events or unique toxicities have not been demonstrated in previous studies, with the exception of a potential risk of allergic reactions in patients with rabbit allergy receiving recombinant human C1INH.

C1INH therapeutic preparations are used to treat hereditary angioedema (HAE). HAE is most often caused by a genetic defect in SERPING1 that leads to loss of C1INH expression or expression of a functionally-deficient C1INH. HAE is defined by recurrent episodes of angioedema without urticaria or pruritus, and treatment with C1INH is able to alleviate these acute symptoms by replacing the deficient or absent C1INH. Long-term prophylaxis with certain C1INH preparations is also used as a treatment for HAE, with the goal of preventing or minimizing the number and severity of angioedema attacks.

C1INH has been identified as being useful for treat other diseases or conditions in which classical pathway complement activity (C1 component) and/or contact system activity (factor XIIa, kallikrein, factor XIa) contributes to undesired immune or inflammatory responses (US 2005/0223416; Caliezi et al. 2000, Pharm. Rev. 52(1):91-112). For example, C1INH has been proposed for use as a therapeutic for reducing ischemia-reperfusion injury (U.S. Pat. No. 8,071,532); as a therapeutic for preventing antibody-mediated rejection of transplanted organs (WO 2015/077543); and as a therapeutic for treating and preventing pre-eclampsia (WO 2019/166556).

### SUMMARY

The disclosure of this application is directed to methods of treating a patient suffering from post-viral neurological symptoms, comprising administering a therapeutically effective amount of a complement inhibitor such as C1 esterase inhibitor (C1INH). In some embodiments, the patient is suffering from or has suffered from neurological symptoms such as extreme fatigue, sensory loss such as loss of taste, cognitive changes, seizures, tremor, and/or stroke. In some embodiments, the patient is a human.

In some embodiments, the method is directed to treating patients suffering from a coronavirus infection. For example, in particular embodiments the coronavirus is SARS-CoV-2. In some embodiments, the patient has antibodies against SARS-CoV-2. In some embodiments, the neurological symptoms are related to the 2019 coronavirus disease (“COVID-19”). In other embodiments, the patient previously had a coronavirus infection. In some embodiments, the patient no longer displays signs of an active infection.

In some embodiments, the C1INH is administered before the neurological symptoms become apparent. In some embodiments, the C1INH is administered after the neurological symptoms become apparent.

In some embodiments, the administered C1INH has an amino acid sequence identical to the amino acid sequence of endogenous human C1INH. In some embodiments, the administered C1INH has at least 70%, at least 75%, at least 80%, at least 85%, at least 90%, at least 95%, at least 96%, at least 97%, at least 98%, or at least 99% identical to the amino acid sequence of endogenous human C1INH. In some embodiments, the C1INH is recombinant human C1INH. In some embodiments, the C1INH has a plasma half-life of less than 6 hours. In some embodiments, the C1INH has a different level of sialic acid residues compared to endogenous plasma-derived human C1INH. In some embodiments, the C1INH is produced in a transgenic animal or in a recombinant cell culture system. In some embodiments, the C1INH is produced in a transgenic rabbit. And in particular embodiments, the C1INH is Ruconest®. Alternatively, in some embodiments, the C1INH is plasma-derived human CI esterase inhibitor.

According to various embodiments of the invention, the C1INH can be administered by a variety of biological routes. For example, in some embodiments, the C1INH is administered intravenously. In some embodiments, the C1INH is administered subcutaneously. And in some embodiments, the C1INH is administered intramuscularly. In some embodiments, the C1INH is self-administered.

The C1INH can be administered at a range of doses and according to a variety of dosing schedules. In some embodiments, the C1INH is administered at a dose of at least about 25 U/kg body weight of the patient. In some embodiments, the C1INH is administered at a dose of at least about 50 U/kg body weight of the patient. In some embodiments, the C1INH is administered at a dose of at least about 60 U/kg body weight of the patient. In some embodiments, the C1INH is administered weekly. In some embodiments, the C1INH is administered weekly for at least eight weeks. In some embodiments, the C1INH is administered at least twice a week. In some embodiments, the C1INH is administered at a dose of about 4200 units. In some embodiments, the patient is administered a pharmaceutical composition comprising C1INH and a pharmaceutically acceptable carrier.

In some embodiments, the C1INH is administered until one or more neuropsychological measures, patient-rated questionnaire, or immunological biomarkers has decreased below its initial pathological status or reached a normal value. In some embodiments, the C1INH is administered until one or more neuropsychological measures has decreased below its initial pathological status or reached a normal value, wherein the neuropsychological measure is selected from the group consisting of the Behavior Rating Inventory of Executive Function-Adult (“BRIEF-A”), Repeatable Battery for the Assessment of Neuropsychological Status (“RBANS”), Beck Depression Inventory II (“BDI II”), and Montreal Cognitive Assessment (“MoCA”). In some embodiments, the C1INH is administered until one or more patient-rated questionnaires reveal improvement from an initial pathological status or reaching a normal status, wherein the patient-rated questionnaire is selected from the group consisting of the Fatigue Severity Scale (“FSS”) questionnaire, Migraine Disability Assessment (“MIDAS questionnaire”), Headache Impact Scale (“HIT”) questionnaire, Activities of Daily Living Sliding Scale and Questionnaire, SF McGill Pain Questionnaire, Gastrointestinal Symptoms Rating Scale (“GSRS”) questionnaire, and Short Form—Quality of Life (“SF-36”) questionnaire. In some embodiments, the C1INH is administered until one or more immunological biomarkers has decreased below its initial pathological status or reached a normal value, wherein the immunological biomarker is selected from the group consisting of a Toll-Like Receptor, glutamic acid decarboxylase 65 (“GAD-65”), the complement protein C4, C1INH, an immunoglobulin and a TH1/TH2 cytokine.

### BRIEF DESCRIPTION OF THE FIGURES

FIGS. 1A and 1B show an exemplary schedule of events for a clinical trial testing the ability of C1INH to treat patients displaying post-viral infection neurological symptoms. In particular, FIGS. 1A and 1B show the activities to be undertaken each week over a 16-week course of administering weekly doses of Ruconest®.

### DETAILED DESCRIPTION

The present disclosure relates to methods of treating viral infection-related neurological symptoms comprising administering C1INH. For example, the disclosure relates to methods of treating post-infectious neurological responses and neurologic changes in patients with SARS-CoV-2 post-viral syndrome.

### I. Definitions of General Terms and Expressions

In order that the present disclosure can be more readily understood, certain terms are first defined. As used in this application, except as otherwise expressly provided herein, each of the following terms shall have the meaning set forth below. Additional definitions are set forth throughout the application. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The terms “administer”, “administering”, “administration”, and the like, as used herein, refer to methods that may be used to enable delivery of a therapeutic, e.g., C1INH, to the desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. Administration refers to the physical introduction of a composition comprising a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, such as orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “C1 Inhibitor,” “C1 esterase Inhibitor,” “C1-INH” and “C1INH” refer to the proteins or fragments thereof that function as serine protease inhibitors to inhibit proteases associated with the complement system, such as proteases C1r and C1s as well as MASP-1 and MASP-2; with the kallikrein-kinin system, such as plasma kallikrein and factor XIIa; and with the coagulation system, such as factor XIa. In addition, C1INH can serve as an anti-inflammatory molecule that reduces the selectins-mediated leukocyte adhesion to endothelial cells. C1INH, as used herein, can be a native serine protease inhibitor or active fragment thereof, or it can comprise a recombinant peptide, a synthetic peptide, peptide mimetic, or peptide fragment that provides similar functional properties—e.g., the inhibition of proteases C1r and C1s, MASP-1, MASP-2, factor XIIa, and/or factor XIa. C1INH, as used herein, includes both plasma-derived C1INH (e.g., purified from human plasma) and recombinantly produced C1INH (e.g., produced in rabbits or cell culture system).

As used herein, the term “viral infection” refers to an infection caused by the presence of a virus in a patient. Symptoms related to a viral infection include both the direct effects of the virus and the effects on or changes within the body that occur as a result of the patient's body (e.g., its immune system) response to the virus.

As used herein, the term “coronavirus” refers to any of the existing or future members of viruses of the family Coronaviridae. One exemplary member of the coronavirus family is the severe acute respiratory syndrome coronavirus (SARS-CoV). Another member of the coronavirus family is the severe acute respiratory syndrome coronavirus 2 (SARS-CoV-2). SARS-CoV-2 is the virus responsible for the 2019 coronavirus disease (COVID-19). Like SARS-CoV and SARS-CoV-2, future members of the coronavirus family may use the Angiotensin-Converting Enzyme 2 (ACE-2) as receptor for cell entry.

The term “neurological symptoms” refers to symptoms that are caused by, or occurring in the nervous system. Exemplary neurological symptoms include, but are not limited to, loss of smell, loss of taste, headaches, fatigue, trouble thinking clearly, cognitive changes, seizures, tremor, and stroke. Such symptoms “become apparent” when they are noticed by the patient, a doctor caring for the patient, or someone else observing the patient. Such symptoms are “clinically recognized” when they have been detected or are reported as part of the patient's medical care.

As used herein, the term “post-viral syndrome” refers to a group of symptoms that occur together after a viral infection, or a condition associated by a set of associated symptoms. For example, SARS-CoV-2 post-viral syndrome is characterized by symptoms such as fatigue, shortness of breath, brain fog and other symptoms even after the patient has recovered from COVID-19. SARS-CoV-2 post-viral syndrome has been associated with neurological symptoms, including extreme fatigue, sensory loss including loss of taste, headaches, cognitive changes, seizures, tremor, and in rare instances, stroke.

Post-viral syndrome in the context of SARS-CoV-2 can take the form of patients suffering from the collection of symptoms known as long COVID syndrome, alternatively referred to as post-acute sequelae of COVID-19 (PASC). Long COVID symptoms include symptoms in pulmonary, nervous, and cardiovascular tissues. Symptoms include sore throat, persistent cough, hoarse voice, shortness of breath, headache, fatigue, brain fog, neuropathy, loss of smell, chest pain, tachycardia, chest tightness, diahhrea, loss of appetite, muscle pain, bone pain, and joint pain.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a cynomolgus monkey. In some embodiments, the subject is a human.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down and/or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” can mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

The term “therapeutically effective amount” refers to an amount of a drug, e.g., C1INH, effective to achieve the desired therapeutic or prophylactic result. A therapeutically effective amount also refers to an amount effective, at dosages and for periods of time necessary, to achieve the desired therapeutic effect. As such, a therapeutically effective amount can be delivered in one or more administrations. A therapeutically effective amount can vary according to factors such as the disease state, age, and weight of the individual. In some instances, the desired result is treating a disease or disorder in a subject. In particular embodiments, the desired result is treating virus-related neurological symptoms.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Any compositions or methods provided herein can be combined with one or more of any of the other compositions and methods provided herein.

### II. C1 Esterase Inhibitors

In the embodiments of the invention, the C1 esterase inhibitor (C1INH) may be any C1INH known to the person skilled in the art. In some embodiments of the invention, the C1INH is a plasma-derived C1INH. In some embodiments of the invention, the C1INH is a recombinant C1INH. In some embodiments of the invention, the C1INH has an amino acid sequence that is identical to the amino acid sequence of human C1INH. In some embodiments, the C1INH has an amino acid sequence that is similar to the amino acid sequence of human C1INH (i.e., having an amino acid sequence at least 90% identical to human C1INH while retaining C1INH's functional activity). In some embodiments, the C1INH has at least 70%, at least 75%, at least 80%, at least 85%, at least 90%, at least 95%, at least 96%, at least 97%, at least 98%, or at least 99% identical to the amino acid sequence of human C1INH, while retaining C1INH's functional activity. The recombinant C1INH can be any recombinant C1INH known the person skilled in the art. It may be produced recombinantly in microbial cells, such as tissue culture cells. The tissue culture cell can be a mammalian tissue culture cell, such as a Chinese Hamster Ovarian (CHO) cell or a human tissue culture cell (see e.g., WO 2016/081889, which is herein incorporated by reference). The recombinant C1INH can be produced in transgenic animals, such as in a transgenic non-human mammal. The recombinant C1INH can be produced in a mouse, goat, bovine, sheep, porcine or an animal from the order Lagomorpha, such as a Leporadae, including a rabbit. In an embodiment, the recombinant C1INH is one produced according to the methods in WO 2001/57079, which is herein incorporated by reference. In some embodiments of the invention, the C1INH is Ruconest®.

In some embodiments of the invention, the C1INH is a modified C1INH as compared to human plasma-derived C1INH. It can be modified to modulate the plasma half-life of the C1INH. A specific modified C1INH is conjugated to enhance the plasma half-life. An exemplary conjugated C1INH to enhance half-life is a conjugated C1INH according to WO 2017/176798, which is herein incorporated by reference. In some embodiments, the conjugated C1INH is a polysialic acid (PSA)-conjugated C1INH, or a polyethylene glycol (PEG)-conjugated C1INH. The modification of the C1INH can be a modified carbohydrate structure as compared to human plasma-derived C1INH. A specific modified C1INH has a reduced level of terminal sialic acid residues as compared to plasma derived C1INH, wherein said reduced level of terminal sialic acid residues may result in a reduction of plasma half-life to less than 6 hours. A specific C1INH having a reduced level of terminal sialic acid residues as compared to plasma derived C1INH is a C1INH according to WO 2001/57079, WO 2004/100982 and WO 2007/073186 which are herein incorporated by reference.

### III. Methods of Administering Pharmaceutical Compositions Comprising C1INH

The C1INH according to the invention can be administered as part of a pharmaceutical composition. In some embodiments, the pharmaceutical composition comprises C1INH and a pharmaceutically acceptable excipient. In some embodiments, the pharmaceutical composition comprises C1INH and a pharmaceutically acceptable carrier. In some embodiments, the pharmaceutical composition comprises C1INH and a pharmaceutically acceptable stabilizer. As such, provided herein are compositions comprising C1INH having the desired degree of purity in a physiologically acceptable carrier, excipient and/or stabilizer. Acceptable carriers, excipients, or stabilizers are nontoxic to recipients at the dosages and concentrations employed. Guidance on pharmaceutically acceptable carriers, excipients, and stabilizers can be found in, for example, Remington's Pharmaceutical Sciences, 22^{nd} ed., Pharmaceutical Press (2012); Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000).

Pharmaceutical compositions according to the invention can be administered by any means known to the person skilled in the art, such as but not limited, to intravenous, transdermal and subcutaneous administration. Intravenous administration is extensively described in WO 2001/57079, WO 2004/100982 and WO 2007/073186. Subcutaneous administration can be performed as in WO 2014/145519, U.S. Pat. No. 9,616,111 B2 and EP 2968434 B1, which are herein incorporated by reference.

As used herein, one unit (U) of C1 esterase inhibitor is the amount of C1INH present in 1 milliliter of human plasma. One such unit corresponds to approximately 275 microgram plasma-derived C1INH. In some embodiments, a therapeutically effective amount of C1INH is administered. The C1INH can be administered in a dose ranging from 25 units/kg body weight to 100 units/kg body weight per administration. In some embodiments, the C1INH can be administered in a dose ranging from about 50 units/kg body weight to about 100 units/kg body weight per administration. Per administration, the dose can be 25 units/kg body weight, 50 units/kg body weight, or 100 units/kg body weight. The total dose per administration can be, for example, 500 units, 600 units 700 units, 800 units, 900 units, 1000 units, 1100 units, 1200 units, 1300 units, 1400 units, 1500 units, 1600 units, 1700 units, 1800 units, 1900 units, 2000 units, 2100 units, 2200 units, 2300 units, 2400 units, 2500 units, 2600 units, 2700 units, 2800 units, 2900 units, 3000 units, 3500 units, 4000 units, 4200 units, 4500 units, 4900 units, 5000 units, 5600 units, 6000 units, 6300 units, 7000 units, 7500 units, 8000 units, 8400 units or 9000 units of C1 inhibitor.

In some embodiments, the compositions comprising C1INH are sterile. Sterile compositions can readily be created, for example, by filtration through sterile filtration membranes.

### IV. Methods of Treating Post-Viral Neurological Symptoms

In some embodiments, the present invention provides methods of treating a patient suffering from neurological symptoms related to a viral infection by administering a therapeutically effective amount of C1INH. The C1INH can be any of the variants of C1INH disclosed herein. In some embodiments, the neurological symptom is one or more of extreme fatigue, sensory loss such as loss of taste, cognitive changes, seizures, tremor, and/or stroke.

In some embodiments, the viral infection is a coronavirus infection. For example, in some embodiments the coronavirus infection is a SARS-CoV infection, a SARS-CoV-2 infection, or MERS-CoV infection. In some embodiments, the coronavirus infection is a coronavirus that uses the Angiotensin-Converting Enzyme 2 (ACE-2) as a receptor for cell entry. In other embodiments, the viral infection is an infection by a virus of unknown origin that induces neurological symptoms.

Embodiments of the invention can begin administration of C1INH at any point after a SARS-CoV-2 or other viral infection. In some embodiments of the invention, treatment begins before neurological symptoms become apparent or are clinically recognized. In some embodiments, treatment begins at the time when, or after, a patient's neurological symptoms become apparent or are clinically recognized. In some embodiments, treatment begins when the SAR-CoV-2 virus or other virus is detectable in the patient's blood. In some embodiments, treatment begins when the SAR-CoV-2 virus or other virus is no longer detectable in the patient.

In some embodiments, C1INH according to the invention is administered to the subject at least once a month, or at least once a week. In some embodiments, the C1INH is administered at least once, twice, three or four times a month. In some embodiments, the C1INH is administered at least once, twice, three, four, five, six or seven times a week. In some embodiments, the C1INH is administered every other day, daily, or twice a day. In some embodiments, after the initial administration the C1INH is administered once every two hours, once every three hours, once every four hours, once every five hours, once every six hours, once every seven hours, once every eight hours, once every nine hours, once every ten hours, once every eleven hours, or once every twelve hours.

In some embodiments, C1INH is administered only once. In some embodiments, C1INH treatment is continued for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or longer as needed. In some embodiments, C1INH treatment is continued until the patient's neurological symptoms cease. In some embodiments, C1INH treatment is continued until the patient's improvement in the neurological symptoms is observed as measured by neuropsychological measures, patient-rated questionnaires, neurological examination, or immunological biomarkers as outlined in Example 1, as Table 1, below.

The medical treatments described herein are methods of treating a patient suffering from neurological symptoms, comprising administering a therapeutically effective amount of C1 esterase inhibitor (C1INH), wherein the neurological symptoms are related to a viral infection. Equivalent formulations intended for use in conjunction with these methods are also described herein. For example, the formulations include a C1 esterase inhibitor (C1INH) for use in the treatment of a patient suffering from neurological symptoms related to a viral infection, wherein the treatment comprises administering a therapeutically effective amount of C1INH to the patient; and use of a C1 esterase inhibitor (C1INH) for the manufacture of a medicament for the treatment of a patient suffering from neurological symptoms, wherein the neurological symptoms are related to a viral infection and the treatment comprises administering a therapeutically effective amount of C1INH to the patient. These equivalent formulations of the embodiments and their features can be used interchangeably.

The methods and formulations described herein are appropriate for use in treating neurological symptoms “related to” a viral infection. Symptoms are related to a viral infection if they, for example, occur as a result of, occur in connection with, occur as a consequence of, or occur after the viral infection. For example, symptoms are related to a viral infection if they are triggered by the immune system's response to the infection. Symptoms are also related to a viral infection if they are more likely to occur in individuals who have had a viral infection.

### Further Embodiments

1. A method of treating a patient suffering from neurological symptoms, comprising administering a therapeutically effective amount of C1 esterase inhibitor (C1INH), wherein the neurological symptoms are related to a viral infection.
2. The method of embodiment 1, wherein the patient is suffering from extreme fatigue, sensory loss such as loss of taste, cognitive changes, seizures, tremor, or stroke.
3. The method of embodiment 2, wherein the patient is suffering from sensory loss.
4. The method of embodiment 3, wherein the patient is suffering from a loss of taste.
5. The method of any one of embodiments 1 to 4, wherein the neurological symptoms are related to COVID-19.
6. The method of any one of embodiments 1 to 5, wherein the viral infection is a coronavirus infection.
7. The method of embodiment 6, wherein the coronavirus is SARS-CoV-2.
8. The method of any one of embodiments 1 to 5, wherein the patient previously had a coronavirus infection.
9. The method of any one of embodiments 1 to 8, wherein the patient has antibodies against SARS-CoV-2.
10. The method of any one of embodiments 1 to 9, wherein the C1INH is administered before the neurological symptoms become apparent or are clinically recognized.
11. The method of any one of embodiments 1 to 9, wherein the C1INH is administered after the neurological symptoms become apparent or are clinically recognized.
12. The method of any one of embodiments 1 to 11, wherein the patient is a human.
13. The method of any one of embodiments 1 to 12, wherein the C1INH has an amino acid sequence at least 70% identity to the amino acid sequence of endogenous human C1INH.
14. The method of any one of embodiments 1 to 13, wherein the C1INH is recombinant human C1INH.
15. The method of any one of embodiments 1 to 14, wherein the C1INH has a plasma half-life of less than 6 hours.
16. The method of any one of embodiments 1 to 15, wherein the C1INH has a different level of sialic acid residues compared to endogenous plasma-derived human C1INH.
17. The method of any one of embodiments 1 to 16, wherein the C1INH is produced in a transgenic animal or in a recombinant cell culture system.
18. The method of embodiment 17, wherein the C1INH is produced in a transgenic rabbit.
19. The method of any one of embodiments 1 to 18, wherein the C1INH is Ruconest®.
20. The method of any one of embodiments 1 to 13, wherein the C1INH is plasma-derived human CI esterase inhibitor.
21. The method of any one of embodiments 1 to 20 wherein the C1INH is administered intravenously.
22. The method of any one of embodiments 1 to 20, wherein the C1INH is administered subcutaneously.
23. The method of any one of embodiments 1 to 20, wherein the C1INH is administered intramuscularly.
24. The method of any one of embodiments 1 to 23, wherein the C1INH is self-administered.
25. The method of any one of embodiments 1 to 24, wherein the C1INH is administered at a dose of at least about 25 U/kg body weight of the patient.
26. The method of any one of embodiments 1 to 25, wherein the C1INH is administered at a dose of at least about 50 U/kg body weight of the patient.
27. The method of any one of embodiments 1 to 26, wherein the C1INH is administered weekly.
28. The method of embodiment 27, wherein the C1INH is administered for at least eight weeks.
29. The method of any one of embodiments 1 to 25, wherein the C1INH is administered at least twice a week.
30. The method of any one of embodiments 1 to 29, wherein the C1INH is administered until one or more neuropsychological measure, patient-rated questionnaire, or immunological biomarker has decreased below its initial pathological status or reached a normal value.
31. The method of embodiment 29, wherein the neuropsychological measure is selected from the group consisting of BRIEF-A, RBANS, BDI II, and MoCA.
32. The method of embodiment 30 or embodiment 31, wherein the patient-rated questionnaire is selected from the group consisting of the FSS questionnaire, MIDAS questionnaire, HIT questionnaire, Activities of Daily Living Sliding Scale and Questionnaire, SF McGill Pain Questionnaire, GSRS questionnaire, and SF-36 questionnaire.
33. The method of any one of embodiments 30 to 32, wherein the immunological biomarker is selected from the group consisting of a Toll-Like Receptor, GAD-65, C4, C1INH, an immunoglobulin or a TH1/TH2 cytokine.
34. The method of any one of embodiments 1 to 33, wherein the C1INH is administered at a dose of about 4200 units.
35. The method of any one of embodiments 1 to 34, wherein the patient is administered a pharmaceutical composition comprising C1INH and a pharmaceutically acceptable carrier.
36. A C1 esterase inhibitor (C1INH) for use in the treatment of a patient suffering from neurological symptoms related to a viral infection, wherein the treatment comprises administering a therapeutically effective amount of C1INH to the patient.
37. The C1INH for use according to embodiment 36, wherein the patient is suffering from extreme fatigue, sensory loss such as loss of taste, cognitive changes, seizures, tremor, or stroke.
38. The C1INH for use according to embodiment 37, wherein the patient is suffering from sensory loss.
39. The C1INH for use according to embodiment 38, wherein the patient is suffering from a loss of taste.
40. The C1INH for use according to any one of embodiments 36 to 39, wherein the neurological symptoms are related to COVID-19.
41. The C1INH for use according to any one of embodiments 36 to 40, wherein the viral infection is a coronavirus infection.
42. The C1INH for use according to embodiment 41, wherein the coronavirus is SARS-CoV-2.
43. The C1INH for use according to any one of embodiments 36 to 42, wherein the patient previously had a coronavirus infection.
44. The C1INH for use according to any one of embodiments 36 to 43, wherein the patient has antibodies against SARS-CoV-2.
45. The C1INH for use according to any one of embodiments 36 to 44, wherein the C1INH is administered before the neurological symptoms become apparent or are clinically recognized.
46. The C1INH for use according to any one of embodiments 36 to 44, wherein the C1INH is administered after the neurological symptoms become apparent or are clinically recognized.
47. The C1INH for use according to any one of embodiments 36 to 46, wherein the patient is a human.
48. The C1INH for use according to any one of embodiments 36 to 47, wherein the C1INH has an amino acid sequence at least 70% identity to the amino acid sequence of endogenous human CIINH.
49. The C1INH for use according to any one of embodiments 36 to 48, wherein the C1INH is recombinant human C1INH.
50. The C1INH for use according to any one of embodiments 36 to 49, wherein the C1INH has a plasma half-life of less than 6 hours.
51. The C1INH for use according to any one of embodiments 36 to 50, wherein the C1INH has a different level of sialic acid residues compared to endogenous plasma-derived human C1INH.
52. The C1INH for use according to any one of embodiments 36 to 51, wherein the C1INH is produced in a transgenic animal or in a recombinant cell culture system.
53. The C1INH for use according to embodiment 52, wherein the C1INH is produced in a transgenic rabbit.
54. The C1INH for use according to any one of embodiments 36 to 53, wherein the C1INH is Ruconest®.
55. The C1INH for use according to any one of embodiments 36 to 48, wherein the C1INH is plasma-derived human CI esterase inhibitor.
56. The C1INH for use according to any one of embodiments 36 to 55, wherein the C1INH is administered intravenously.
57. The C1INH for use according to any one of embodiments 36 to 55, wherein the C1INH is administered subcutaneously.
58. The C1INH for use according to any one of embodiments 36 to 55, wherein the C1INH is administered intramuscularly.
59. The C1INH for use according to any one of embodiments 36 to 58, wherein the C1INH is self-administered.
60. The C1INH for use according to any one of embodiments 36 to 59, wherein the C1INH is administered at a dose of at least about 25 U/kg body weight of the patient.
61. The C1INH for use according to any one of embodiments 36 to 60, wherein the C1INH is administered at a dose of at least about 50 U/kg body weight of the patient.
62. The C1INH for use according to any one of embodiments 36 to 61, wherein the C1INH is administered weekly.
63. The C1INH for use according to embodiment 62, wherein the C1INH is administered for at least eight weeks.
64. The C1INH for use according to any one of embodiments 36 to 60, wherein the C1INH is administered at least twice a week.
65. The C1INH for use according to any one of embodiments 36 to 64, wherein the C1INH is administered until one or more neuropsychological measure, patient-rated questionnaire, or immunological biomarker has decreased below its initial pathological status or reached a normal value.
66. The C1INH for use according to embodiment 64, wherein the neuropsychological measure is selected from the group consisting of BRIEF-A, RBANS, BDI II, and MoCA.
67. The C1INH for use according to embodiment 65 or embodiment 66, wherein the patient-rated questionnaire is selected from the group consisting of the FSS questionnaire, MIDAS questionnaire, HIT questionnaire, Activities of Daily Living Sliding Scale and Questionnaire, SF McGill Pain Questionnaire, GSRS questionnaire, and SF-36 questionnaire.
68. The C1INH for use according to any one of embodiments 65 to 67, wherein the immunological biomarker is selected from the group consisting of a Toll-Like Receptor, GAD-65, C4, C1INH, an immunoglobulin or a TH1/TH2 cytokine.
69. The C1INH for use according to any one of embodiments 36 to 68, wherein the C1INH is administered at a dose of about 4200 units.
70. The C1INH for use according to any one of embodiments 36 to 69, wherein the patient is administered a pharmaceutical composition comprising C1INH and a pharmaceutically acceptable carrier.
71. Use of a C1 esterase inhibitor (C1INH) for the manufacture of a medicament for the treatment of a patient suffering from neurological symptoms, wherein the neurological symptoms are related to a viral infection and the treatment comprises administering a therapeutically effective amount of C1INH to the patient
72. The use according to embodiment 71, wherein the patient is suffering from extreme fatigue, sensory loss such as loss of taste, cognitive changes, seizures, tremor, or stroke.
73. The use according to embodiment 72, wherein the patient is suffering from sensory loss.
74. The use according to embodiment 73, wherein the patient is suffering from a loss of taste.
75. The use according to any one of embodiments 71 to 74, wherein the neurological symptoms are related to COVID-19.
76. The use according to any one of embodiments 71 to 75, wherein the viral infection is a coronavirus infection.
77. The use according to embodiment 76, wherein the coronavirus is SARS-CoV-2.
78. The use according to any one of embodiments 71 to 77, wherein the patient previously had a coronavirus infection.
79. The use according to any one of embodiments 71 to 78, wherein the patient has antibodies against SARS-CoV-2.
80. The use according to any one of embodiments 71 to 79, wherein the C1INH is administered before the neurological symptoms become apparent or are clinically recognized.
81. The use according to any one of embodiments 71 to 79, wherein the C1INH is administered after the neurological symptoms become apparent or are clinically recognized.
82. The use according to any one of embodiments 71 to 81, wherein the patient is a human.
83. The use according to any one of embodiments 71 to 82, wherein the C1INH has an amino acid sequence at least 70% identity to the amino acid sequence of endogenous human C1INH.
84. The use according to any one of embodiments 71 to 83, wherein the C1INH is recombinant human C1INH.
85. The use according to any one of embodiments 71 to 84, wherein the C1INH has a plasma half-life of less than 6 hours.
86. The use according to any one of embodiments 71 to 85, wherein the C1INH has a different level of sialic acid residues compared to endogenous plasma-derived human C1INH.
87. The use according to any one of embodiments 71 to 86, wherein the C1INH is produced in a transgenic animal or in a recombinant cell culture system.
88. The use according to embodiment 87, wherein the C1INH is produced in a transgenic rabbit.
89. The use according to any one of embodiments 71 to 88, wherein the C1INH is Ruconest®.
90. The use according to any one of embodiments 71 to 83, wherein the C1INH is plasma-derived human CI esterase inhibitor.
91. The use according to any one of embodiments 71 to 90, wherein the C1INH is administered intravenously.
92. The use according to any one of embodiments 71 to 90, wherein the C1INH is administered subcutaneously.
93. The use according to any one of embodiments 71 to 90, wherein the C1INH is administered intramuscularly.
94. The use according to any one of embodiments 71 to 93, wherein the C1INH is self-administered.
95. The use according to any one of embodiments 71 to 94, wherein the C1INH is administered at a dose of at least about 25 U/kg body weight of the patient.
96. The use according to any one of embodiments 71 to 95, wherein the C1INH is administered at a dose of at least about 50 U/kg body weight of the patient.
97. The use according to any one of embodiments 71 to 96, wherein the C1INH is administered weekly.
98. The use according to embodiment 97, wherein the C1INH is administered for at least eight weeks.
99. The use according to any one of embodiments 71 to 95, wherein the C1INH is administered at least twice a week.
100. The use according to any one of embodiments 71 to 99, wherein the C1INH is administered until one or more neuropsychological measure, patient-rated questionnaire, or immunological biomarker has decreased below its initial pathological status or reached a normal value.
101. The use according to embodiment 99, wherein the neuropsychological measure is selected from the group consisting of BRIEF-A, RBANS, BDI II, and MoCA.
102. The use according to embodiment 100 or embodiment 101, wherein the patient-rated questionnaire is selected from the group consisting of the FSS questionnaire, MIDAS questionnaire, HIT questionnaire, Activities of Daily Living Sliding Scale and Questionnaire, SF McGill Pain Questionnaire, GSRS questionnaire, and SF-36 questionnaire.
103. The use according to any one of embodiments 100 to 102, wherein the immunological biomarker is selected from the group consisting of a Toll-Like Receptor, GAD-65, C4, C1INH, an immunoglobulin or a TH1/TH2 cytokine.
104. The use according to any one of embodiments 71 to 103, wherein the C1INH is administered at a dose of about 4200 units.
105. The use according to any one of embodiments 71 to 104, wherein the patient is administered a pharmaceutical composition comprising C1INH and a pharmaceutically acceptable carrier.

### EXAMPLES

### Example 1

### Clinical Trial Testing the Effects of C1INH in COVID-19 Patients Suffering From Post-COVID-19-Related Neurological Symptoms

C1 esterase inhibitor (C1INH) is the direct natural inhibitor of both the complement and the lectin pathway activation and is the most potent known natural inhibitor of this inflammation cascade. We hypothesize that administration of Ruconest® will counteract the deleterious complement activation that occurs as a part of post-COVID-19 neuronal symptoms. To analyse the effects of C1INH on symptoms resulting from SARS-CoV-2 infection and the resulting COVID-19 disease, a double-blinded, randomized, placebo-controlled, cross-over, clinical trial will be performed testing the efficacy of Ruconest®. This trial will include up to a 2 week screening phase, 2 month treatment or placebo phase, 2 month cross-over to treatment or placebo phase, and 1 week post-treatment visit, considered end of study visit (EOS). Forty participants, 18 years of age and older may participate.

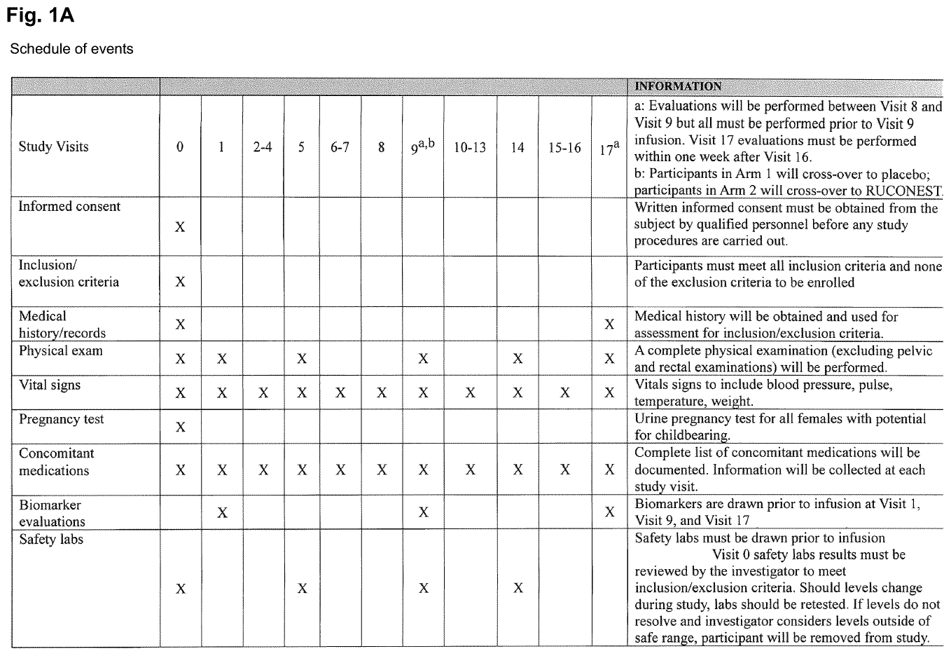

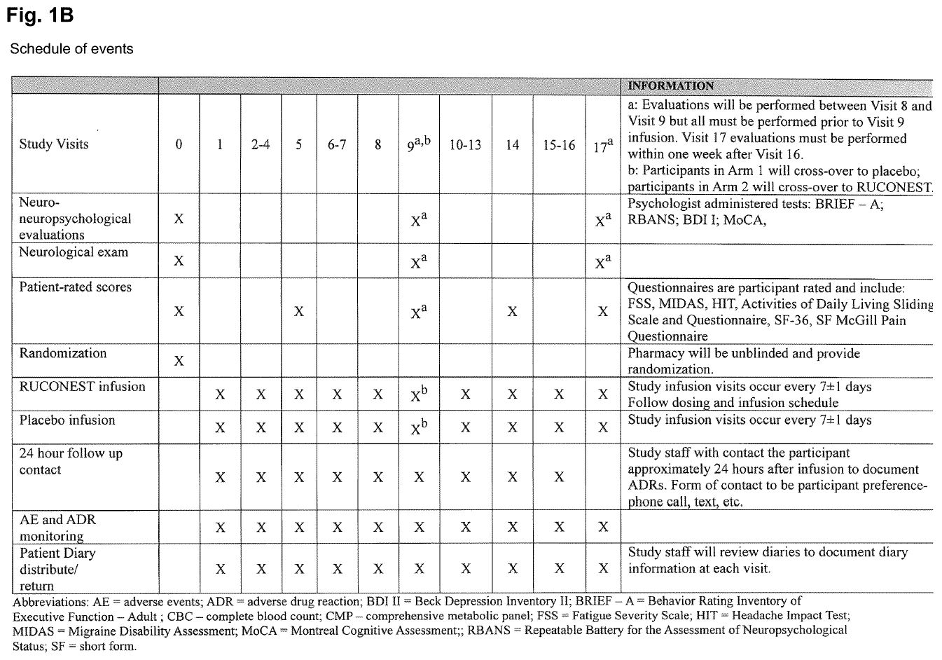

There will be 2 arms containing a total of approximately 40 participants. Participants will be randomized into the arms in 1:1 ratio. In arm 1, participants will receive IV Ruconest® once a week for 8 weeks and then crossover to receive placebo once a week for 8 weeks. In arm 2, participants will receive placebo once a week for 8 weeks and then cross-over to receive Ruconest® once a week for 8 weeks. The total length of study participation will be approximately 19 weeks. A schedule of events for this trial is summarized in FIGS. 1A and 1B.

### Objectives

The primary objectives of this study will be to evaluate the potential benefit of Ruconest® in neuroimmune disorders related to SARS-CoV-2 post-viral fatigue syndrome.

The outcome of the study will be measured by the following neuropsychological measures, patient-rated questionnaires, neurological examination, and immunological biomarkers. These outcome measures are specified in Table 1 below.

TABLE 1

| Category | Outcome Measures |
|---|---|
| Neuropsychological | Behavior Rating Inventory of Executive |
| measures | Function-Adult (BRIEF-A) |
| | Repeatable Battery for the Assessment of |
| | Neuropsychological Status (RBANS) |
| | Beck Depression Inventory II (BDI II) |
| | Montreal Cognitive Assessment (MoCA) |
| Patient-rated | Fatigue Severity Scale (FSS) |
| questionnaires | Migraine Disability Assessment (MIDAS) |
| | Headache Impact Scale (HIT) |
| | Activities of Daily Living Sliding Scale and |
| | Questionnaire |
| | SF McGill Pain Questionnaire |
| | Gastrointestinal Symptoms Rating Scale |
| | (GSRS) |
| | SF-36 |
| Neurological | Complete neurological exam |
| examination | |
| Immunological | Toll-Like Receptor Function Assay |
| biomarkers | GAD-65 |
| | Complement Panel (C4, CI-INH, CI-INH |
| | Function) |
| | Immunoglobulins, Quantitative, IgA, IgE, |
| | IgG, IgM |
| | Immunoglobulins G, subclasses (1-4) |
| | TH1/TH2 Cytokine Levels |

Immunological biomarker laboratory tests and safety laboratory tests will be performed according to the schedule listed in Table 2 below.

TABLE 2

| Visit | Tests Performed |
|---|---|
| 1 | Immunoglobulins, Quantitative, IgA, IgE, IgG, IgM |
| | Immunoglobulins G, subclasses (1-4) |
| 1, 9, and 17 | GAD-65 |
| | Toll-Like Receptor Function Assay |
| | Complement Panel (C4, CI-INH, and CI-INH |
| | Function) |
| | TH1/TH2 Cytokine Panel |
| 0, 5, 9, and 14 | CBC with Differential & Platelet Count |
| | Comprehensive Metabolic Panel |
| Immunological | Toll-Like Receptor Function Assay |
| biomarkers | GAD-65 |
| | Complement Panel (C4, CI-INH, CI-INH Function) |
| | Immunoglobulins, Quantitative, IgA, IgE, IgG, IgM |
| | Immunoglobulins G, subclasses (1-4) |
| | TH1/TH2 Cytokine Levels |

The Behavior Rating Inventory of Executive Function, Adult Version (BRIEF-A) is a standardized measure that captures views of an adult's executive functions or self-regulation in his or her everyday environment. Both a self-report and an informant report are used. There are nine nonoverlapping theoretically and empirically derived clinical scales: Inhibit, Self-Monitor, Plan and Organize, Shift, Initiate, Task Monitor, Emotional Control, Working Memory, and Organization of Materials. It is useful for a wide variety of developmental, systemic, neurological, and psychiatric disorders and includes two broad indexes, three validity scales, and a summary score.

The Repeatable Battery for the Assessment of Neuropsychological Status (RBANS, versions A, B and C) provides an individually administered battery to detect and monitor cognitive decline and overall neurocognitive status. The instrument enables clinicians to assess multiple domains, including Attention, Immediate and Delayed Memory, Language, and Visuospatial/Constructional.

The Beck Depression Inventory II (BDI II) is an internationally recognized, self-administered 21-item self-report scale, presented in multiple choice format, designed to detect presence of depression in adults, to measure characteristic attitudes and symptoms of depression independent of any particular theoretical bias. Each of the inventory items correspond to a specific category of depressive symptom and/or attitude. Each category purports to describe a specific behavioral manifestation of depression and consists of a graded series of four self-evaluative statements. The statements are rank ordered and weighted to reflect the range of severity of the symptom from neutral to maximum severity. Numerical Values of zero, one, two, or three are assigned to each statement to indicate degree of severity. Adding up the scores for all the twenty-one questions, the single total score is produced indicating intensity of the depression.

The Montreal Cognitive Assessment (MoCA, versions 8.0, also 7.1, 7.2) was designed as a screening instrument for cognitive dysfunction. It assesses different cognitive domains: Attention and Concentration, Executive functions, Memory, Language, Visuoconstructional skills, Conceptual thinking, Calculations, and Orientation.

### Study Population

Approximately 40 patients suffering from post-COVID-19 neurological symptoms will be enrolled. To be eligible, patients must be age 18 or older, had a previous confirmed diagnosis of SARS-CoV-2, be experiencing SARS-CoV-2 post-viral fatigue syndrome four weeks after recovery from SARS-CoV-2 infection, be experiencing neurological symptoms including fatigue, and be willing to comply with all aspects of the protocol, including blood draws.

Exclusion criteria include receiving any form of C1-INH therapy either acute or prophylactic treatment, a history or suspicion of allergy to rabbits, neurological conditions related to injury, neuropathy related to diabetes, current pregnancy or lactation, being largely incapacitated or bed ridden, being enrolled in any other clinical study judged not to be scientifically or medically compatible with this study, and patients who, in the investigator's opinion, might not be suitable for the trial for safety reasons.

### Treatment

After consent, participants will be in the screening phase (up to 2 weeks—Visit 0). Before treatment phase, participants will be randomized using a block method by the pharmacy for a 1:1 ratio. During the treatment phase, participants will receive Ruconest® or placebo IV once a week for 8 weeks followed by a cross-over treatment phase for an additional 8 weeks. Visit 17 is scheduled within one week after Visit 16 infusion. Total length of subject participation is approximately 19 weeks.

Ruconest® is purified from the milk of rabbits expressing the gene coding for human C1INH. Ruconest® is supplied as a sterile, preservative-free, white/off-white lyophilized powder for reconstitution for injection. Each vial contains 2100 units of Ruconest®, 937 mg of sucrose, 83.3 mg of sodium citrate dihydrate and 1.0 mg of citric acid monohydrate. One international unit (U) of C1INH activity is defined as the equivalent of C1INH activity present in 1 mL of pooled normal plasma. After reconstitution with 14 mL of sterile water for injection, each vial contains 150 U of Ruconest per 1 mL in a 20 mM sodium citrate buffer with a pH of 6.8; vials are for single use only.

Ruconest® will be administered as slow (5 min) intravenous injection via a peripheral or central intravenous line. A uniform dose of 4200 U was chosen irrespective of body weight. The licensed dosage for C1INH is weight-based (50 U/kg up to 84 kg and 4200 U for a bodyweight of ≥84 kg). This is based on the aim to at least achieve a level of 0.7 U/ml C1INH in patients (lower limit of normal of C1INH activity). Simulation studies have revealed that this aim is achievable with the licensed dosing. However, these simulations have also shown that C1INH levels will be lower when using 50 U/kg compared to 4200 U in patients with a bodyweight <84 kg. Saline will be used to flush after infusion of Ruconest®.

Placebo will be sterile saline given at the same volume as Ruconest® and using the same pumps and rates.

### Statistical Methods

This study is a proof-of-concept (PoC) study, and it has to show positive results only exploratorily and will use descriptive statistical methods. The study endpoints will be defined as the average of all values recorded during the screening phase, treatment period and during the last observation. The following descriptive statistics will be provided: number of observations, mean, standard deviation, standard error, median, interquartile range, variance, minimum and maximum. Trends in these values during the treatment period will be analyzed to determine correlations or efficacies. A more detailed statistical analysis, including secondary efficacy analyses and exploratory sub-analyses, may be used upon completion for further analysis.

Having now fully described this invention, it will be understood by those of ordinary skill in the art that the same can be performed within a wide and equivalent range of conditions, formulations, and other parameters without affecting the scope of the invention or any embodiment thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1.-15. (canceled)

16. A method for treating a patient suffering from neurological symptoms related to a viral infection, the method comprising administering a therapeutically effective amount of a C1 esterase inhibitor (C1INH) to the patient.

17. The method according to claim 16, wherein the patient is suffering from extreme fatigue, sensory loss such as loss of taste, cognitive changes, seizures, tremor, or stroke.

18. The method according to claim 16, wherein the neurological symptoms are related to COVID-19 and/or wherein the viral infection is a coronavirus infection, wherein the coronavirus preferably is SARS-CoV-2.

19. The method according to claim 16, wherein the patient previously had a coronavirus infection and/or wherein the patient has antibodies against SARS-CoV-2.

20. The method according to claim 16, wherein the C1INH is administered before the neurological symptoms become apparent or are clinically recognized or wherein the C1INH is administered after the neurological symptoms become apparent or are clinically recognized.

21. The method according to claim 16, wherein the patient is a human and/or wherein the C1INH has an amino acid sequence at least 70% identity to the amino acid sequence of endogenous human C1INH.

22. The method according to claim 16, wherein the C1INH is recombinant human C1INH and/or wherein the C1INH has a plasma half-life of less than 6 hours and/or wherein the C1INH has a different level of sialic acid residues compared to endogenous plasma-derived human C1INH.

23. The method according to claim 16, wherein the C1INH is produced in a transgenic animal, preferably a transgenic rabbit, or in a recombinant cell culture system.

24. The method according to claim 16, wherein the C1INH is Ruconest® and/or wherein the C1INH is plasma-derived human Cl esterase inhibitor.

25. The method according to claim 16, wherein the C1INH is administered intravenously, subcutaneously and/or intramuscularly and/or wherein the C1INH is self-administered.

26. The method according to claim 16, wherein the C1INH is administered at a dose of at least about 25 U/kg body weight of the patient, or wherein the C1INH is administered at a dose of at least about 50 U/kg body weight of the patient.

27. The method according to claim 16, wherein the C1INH is administered weekly and/or wherein the C1INH is administered for at least eight weeks and/or wherein the C1INH is administered at least twice a week.

28. The method according to claim 16, wherein the C1INH is administered until one or more neuropsychological measure, patient-rated questionnaire, or immunological biomarker has decreased below its initial pathological status or reached a normal value, wherein the neuropsychological measure is preferably selected from the group consisting of BRIEF-A, RBANS, BDI II, and MoCA, wherein the patient-rated questionnaire is preferably selected from the group consisting of the FSS questionnaire, MIDAS questionnaire, HIT questionnaire, Activities of Daily Living Sliding Scale and Questionnaire, SF McGill Pain Questionnaire, GSRS questionnaire, and SF-36 questionnaire, and wherein the immunological biomarker is preferably selected from the group consisting of a Toll-Like Receptor, GAD-65, C4, C1INH, an immunoglobulin or a TH1/TH2 cytokine.

29. The method according to claim 16, wherein the C1INH is administered at a dose of about 4200 units.

30. The method according to claim 16, wherein the patient is administered a pharmaceutical composition comprising C1INH and a pharmaceutically acceptable carrier.
